(19) United States
(12) Patent Application Publication
(10) Pub. No.: US 2018/0045711 A1
(43) Pub. Date: Feb. 15, 2018

(54) METHOD AND DEVICE FOR DETECTING IN REAL TIME A SECRETED COMPOUND AND THE SECRETION TARGET, AND USES THEREOF

(71) Applicants: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR)

(72) Inventors: Yoann ROUPIOZ, Goncelin (FR); Loic LEROY, Grenoble (FR); Patrice MARCHE, Meylan (FR); Olivier DELLEA, La Talaudiere (FR); Myriam CUBIZOLLES, Corenc (FR); Cyril HERRIER, Yffiniac (FR); Thierry LIVACHE, Jarrie (FR)

(73) Assignees: COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR)

(21) Appl. No.: 15/555,777

(22) PCT Filed: Mar. 4, 2016
(86) PCT No.: PCT/EP2016/054685

§ 371 Date: Sep. 5, 2017

(30) Foreign Application Priority Data
Mar. 6, 2015 (FR) 1551918

(51) Int. Cl. G01N 33/50; G01N 33/543; G01N 33/53

(52) U.S. Cl. CPC G01N 33/5005; G01N 33/5302; G01N 33/54373; G01N 33/6863

(57) ABSTRACT

A method for measuring in real time a secretion of a compound by a target, and a device implementing the method, the method including: culturing, in a liquid medium, in a culture chamber, a plurality of targets including at least one target, the culture chamber including: (i) at least one 1^{st} surface on which the target is present, presence of the target generating a 1^{st} signal; and (ii) at least one 2^{nd} surface that is different from the 1^{st} surface and not coplanar with the 1^{st} surface, which surface is functionalized by at least one ligand specifically binding to the compound secreted by the target, the specific bond between the ligand and the compound generating a 2^{nd} signal that is distinct from the 1^{st} signal; and detecting in real time the 2^{nd} signal and optionally the 1^{st} signal.

### TECHNICAL FIELD

The present invention relates to the field of methods and devices for detection applied to biology and notably the field of multiple detection applied to biology.

Indeed, the present invention relates to a method and particular devices giving the possibility of not only determining and quantifying the cell secretions but also of identifying the relevant cell type(s), the uses of such method and devices as well as the methods for preparing these devices.

### STATE OF THE PRIOR ART

The communication between cells is a biological event of prime importance. The communication modes may have different forms like a physical contact between cells or else the emission of chemical compounds by a cell and the reception of these compounds by another cell.

The latter mode corresponds to a chemical communication mode in which the information of the message is borne by a set of parameters such as the nature, the amount, the frequency and the period for secretion of said compounds, the cell type of the emitting cell, the cell type of the receiving cell (or “target” cell). Thus, the secreting activities of the cells are subject to many biomedical investigations for, for example tracking and comprising the set of immune cascades involved in the development of infections, of cancers, of self-immune diseases, of allergies, of the impact of vaccines or in the treatment of graft rejections. The possibility of following the triggering of an immune response is therefore a significant challenge for many diagnostic and therapeutic biomedical applications.

The whole of the techniques described to this day resorting to a sequential protocol which comprises an incubation, and then a visualization and an end-point readout, there does not exist any rapid test giving the possibility of detecting cell secretions in a few minutes, or even in a few hours. On the other hand, the (fluorescent and/or colorimetric disclosure) labeling steps also limit the number of parameters (search for different cytokines for example) which may be followed from a same sample (problem of overlapping of the spectra in the visible spectrum and/or excitation/emission of UVs or deconvolution of signals consisting of different colors). Finally, a corollary of these indispensable labeling steps is the impossibility to follow, in real time, the secretion kinetics in the extracellular medium.

A significant point to be underlined in the implementation of the presently available techniques is the absolute necessity of passing through labeling steps and a posteriori readout, i.e. as an end-point. This mode for producing a result does not give any information in real time on the secretions and therefore does not give the possibility of accessing the kinetic parameters of the interactions such as association, and dissociation constants, kinetic response profiles according to the cytokines and/or of the cell types . . . .

At the present time, several analysis devices give the possibility of determining/quantifying the cell secretions stemming from one or several cell types.

Among these analysis devices, it is possible to mention the diagnostic test T-SPOT®.TB (Oxford Immunotec) notably described in the international application WO 98/23960 [1] which allows the analysis of the immune response at a cellular scale for diagnosing tuberculosis. This test is based on the so called ELISPOT technique [2] which involves culturing, in multi-well plates, cell samples for time intervals from a few hours to a few days, followed by washing of these wells (removal of the secreting cells), and by a succession of washing/visualization steps (ELISA sandwich test). Since the bottom of these wells is covered with specific antibodies of a secreted compound, a molecular assembly by means of a second antibody followed by a colorimetric visualization gives the possibility of viewing a posteriori “spots” at the location where a cell has settled during the culture and has secreted a compound in the culture medium. The diffusion of the compounds in the environment close to a cell therefore gives the possibility of revealing characteristic spots of an individual cellular secreting activity.

Other devices apply techniques using flow cytometry.

Thus, in the ICS technique for “IntraCellular Staining”, the cell membranes are permeabilized for marking the cytokines inside the cell by means of antibodies coupled with fluorophores [3]. This type of manipulations followed by a passage in flow cytometry gives the possibility of an individual analysis of the cells and may give the possibility of sorting/selecting the cells. On one hand, the permeabilization of the membranes significantly affects the viability of the cells, and prevents any subsequent putting back into a culture of selected cells. Further, in this system, the measurement detects the presence of the cytokine inside the cell without being able to state whether the latter is actually secreted. In other words, this technique only provides an indicative datum since the presence of a protein in the intracellular medium does not systematically preclude its presence in the extracellular medium.

The technique for encapsulating the cells in agarose gels functionalized with antibody specific to the secreted molecules is also based on an analysis in flow cytometry by detection of fluorescence emitted by sandwiched molecular assemblies [4]. Gelled beads containing a secreting cell are then fluorescent and may be sorted by flow cytometry.

The test of Miltenyi Biotec is based on the combination of four different antibodies giving the possibility of cross examining the characteristic information of the secreting cell type and of the cytokine released in the extracellular membrane. Before their individual analysis in flow cytometry, the cells are labeled at their surface with an antibody specific of a membrane marker allowing identification of their cell type. This antibody is itself conjugated covalently to a second antibody, forming a chimeric antibody, specific of the cytokine of interest. If the latter is secreted in the extracellular medium, it is then captured by this second antibody on which a third antibody (specific of another epitope of the same cytokine) may be complexed and revealed by the recognition of a fourth specific anti-species antibody of the immunoglobulins produced in the host species of the third antibody [5]. This approach however suffers from a complex and costly application in particular in the case of simultaneous search for different cytokines.

The “patch-clamp” technique which allows quantification, by capacitance and/or ampere measurement of the electrically charged compounds, secreted by the portion of the plasma membrane obstructing the pipette is not applicable for simultaneous study of a great number of cells [6]. Above all this is to study the physico-chemical processes involved during the secretion at a cellular scale and notably at a macromolecular scale of the membrane.

Mention may further be made of other methods allowing determination and optionally quantification of the cell secretions issued from one or several cell types such as:
electrochemical methods [7] which, although very sensitive, may be complex and expensive to implement;
methods involving a microfluidic subdivision as described in the international application WO 2011/056643 [8] wherein individual cells are introduced by microfluidic handling in reaction microchambers;
methods involving a transmission opto-fluidic platform [9] with plasmon analysis simultaneously coupled with electrochemical analysis; these methods however do not give the possibility of differentiating the cells and the biological validity of the results may be distorted because the in vivo environment of the biological material is not very or not simulated; and
methods involving a phenotyping platform; the international application WO 2006/060646 [10] proposes a gene characterization of secretions and an analysis of the gene regulation mechanism internal to the cell(s); these methods however do not allow an analysis in real time and the biological validity of the results may be distorted because the in vivo environment of the biological material is rarely or not simulated.

Finally, the device described in [11] implements the surface plasmon resonance imaging (SPRi) and an analysis support consisting of a glass prism covered on one face by a gold layer on which are found one or several type(s) of ligands corresponding to a detection surface. The principle consists of illuminating, through the prism and under different angles (variation of the theta angle), the detection surface with a polarized beam and of analyzing the variations of the reflected beam caused by the interaction of the evanescent wave generated by plasmon resonance and the analyzed medium. The penetration length of the evanescent wave being of the order of a few hundred nm, the method is then only sensitive to variations of refractive index of the medium, very close to the gold surface (i.e., at the specific or non-specific adsorption of the molecules at the functional surface of the gold). The simultaneous analysis of a surface of 1 cm² by imaging allows the observation of specific patterns with different components. However, in this configuration and in the one described in the patent application US 2013/137085 [12], the signal of the cells is superposed to the signal of the secretions, which may be detrimental to the quality of the analysis because of the absence of discrimination between both signals.

Further, up to today, the analysis of the secretome i.e. the medium containing the secretions but not cells is often moved away from the area for recognizing the cells, causing the impossibility of associating the cell type and the secretion [13].

In order to summarize, the tools, i.e. the methods and the devices, available to this day are quasi all dedicated to single-parameter or multi-parameter measurements with a single type of secretion sought as an end-point and this, with a very low throughput.

The present inventors therefore set their goal of proposing industrializable method and device, giving the possibility of determining and of detecting in a simple, rapid way and in real time, both the secreted compound and the secreting cell and this without any loss in sensitivity.

### DISCUSSION OF THE INVENTION

The present invention gives the possibility of solving technical problems and drawbacks listed earlier. Indeed, the inventors propose a method and devices giving the possibility of tracking, in real time cell secretions by a group of cells or individual cells. Such a method gives access to two new properties of cell secretion—in addition to the qualitative and quantitative characterization—which are, on the one hand, the tracking of response kinetics and, on the other hand, of intercellular variation within a complex sample.

In particular, the present invention proposes a method for measuring in real time the secretion of a compound Cₒ by a target Cᵢ, said method comprising:
the culture in a liquid medium in a culture chamber, of a plurality of targets among which is found at least one target Cᵢ, said culture chamber having:

i) at least one 1^{st} surface on which said target Cᵢ is present, the presence of said target Cᵢ on said 1^{st} surface generating a 1^{st} signal, and

ii) at least one 2^{nd} surface, different from said 1^{st} surface and non-coplanar with said 1^{st} surface, functionalized with at least one ligand specifically binding to said compound Cₒ secreted by said target Cᵢ, the specific binding between said ligand and said compound Cₒ generating a 2^{nd} signal distinct from said 1^{st} signal, and
the detection in real time of said 2^{nd} signal and optionally the detection of said 1^{st} signal.

In such a method for measuring in real time the secretion of a compound Cₒ by a target Cᵢ, the target Cᵢ may not be bound to the 1^{st} surface and just deposited or settled on the latter. Alternatively, the 1^{st} surface may be functionalized with at least one probe specifically binding to said target Cᵢ.

Thus, the present invention proposes a method for measuring in real time the secretion of a compound Cₒ by a target Cᵢ, said method comprising:
the culture in a liquid medium in a culture chamber of a plurality of targets among which is found at least one target Cᵢ, said culture chamber having:

i) at least one 1^{st} surface functionalized with at least one probe specifically binding to said target Cᵢ, the specific binding between said probe and said target Cᵢ generating a 1^{st} signal, and
ii) at least one 2^{nd} surface, different from said 1^{st} surface and non-coplanar with said 1^{st} surface, functionalized by at least one ligand specifically binding to said compound Cₒ secreted by said target Cᵢ bound to said probe, the specific binding between said ligand and said compound Cₒ generating a 2^{nd} signal distinct from said 1^{st} signal, and
the detection in real time of said 2^{nd} signal and optionally the detection of said 1^{st} signal.

Within the scope of the present invention, by <<target>> is meant a secreting element comprising one or several identical or different cells. Thus, a target may be (i) an individual cell, (ii) a set of identical cells such as a group of encapsulated identical cells, (iii) a set of cells of at least two different types such as a tissue possibly stemming from biopsy, from biopsy by drilling/suction with a thin needle (or FNAB for <<Fine Needle Aspiration Biopsy>>), from a macrobiopsy or from a microbiopsy, a tissue fragment possibly stemming from a biopsy, from a biopsy with drilling/aspiration with a thin needle (or FNAB for <<Fine Needle Aspiration Biopsy>>), from a macrobiopsy, from a microbiopsy or further (iv) a set of cells of at least two encapsulated different types. Within the scope of the present invention, by <<tissue>> is meant both a natural tissue and a synthetic or artificial tissue of the artificial skin type.

Thus, in the present invention, the expression <<one target>> may be used interchangeably, either with the expression <<one cell>>, or with the expression <<cells>>.

Within the scope of the present invention, by <<cell>> is meant both a cell of the prokaryote type or of the eukaryotic type. Among the eukaryotic cells, the cell or the cells may be a yeast such as a yeast of the genus Saccharomyces or Candida, a fungi cell, an algae cell, a plant cell or an animal cell such as a mammal cell, a fish cell or an insect cell. The mammal cells may notably be tumoral cells, immortalized cell lines, somatic or geminal cells or stem cells. In a non-exclusive way these may be endocrine cells, exocrine cells, red blood cells, osteoblasts, neuronal cells, nerve cells, epithelial cells, hepatocytes, muscle cells, lymphocytes B, lymphocytes T, caliciform cells, chromaffin cells, cells of granulosa, alpha or beta cells of Langerhans islets, progenitor cells or cells infected with an infectious agent such as a virus.

The cells of the prokaryotic type are bacteria which may be of the Gram positive type or Gram negative type, or archaea. Among these bacteria, mention may be made, as an example and in a non-exhaustive way, bacteria belonging to branches of spirochetes and chlamydiae, of bacteria belonging to the families of enterobacteria, streptococcuses, micrococci, legionellas, mycobacteria, bacillaceae, cyanobacteria and other bacteria. From among archaeal bacteria, mention may be made, as examples and in a non-exhaustive way, the archaeal bacteria belonging to the phyla of Crenarchaeotes and Euryarchaeotes.

The cells implemented within the scope of the present invention may be obtained from a primary cell culture or a culture of a cell line, from a tissue or a tissue section possibly stemming from a biopsy, from a biopsy by drilling/aspiration with a fine needle (or FNAB for “fine needle aspiration biopsy”), from a macrobiopsy or from a microbiopsy, or from a sample stemming from a fluid such as a biological fluid; a sample in a culture medium or in a biological culture reactor like a cell culture; a sample in a food matrix, preferably diluted in a buffer; a sample in a chemical reactor; a sample in a water treatment plant; a sample in a composting station; tap water, water from rivers, ponds, lakes, the sea, pools, or aero-refrigerated towers or of subterranean origin; a sample from a liquid industrial effluent; waste water notably stemming from intensive breeding or industries from the chemical, pharmaceutical or cosmetic field or a sample stemming from air filtration, said sample may have undergone preliminary different treatments like centrifugation, concentration, dilution, encapsulation . . . .

Within the scope of the present invention, by “sample” is meant any type of sample collection, for example, by contact, scraping, drilling, draining, washing, rinsing, suction, pumping, etc. . . .

The biological fluid is advantageously selected from the group consisting of blood such as full blood or anti-coagulated full blood, blood plasma, lymph, saliva, tears, sperm, urine, feces, milk, cerebrospinal fluid, interstitial liquid, synovial liquid, an isolated fluid from bone marrow, a mucus or fluid from the respiratory, intestinal or Benito-urinary tract, from extracts of tissues and from extracts of organs. Thus, the biological fluid may be any fluid naturally secreted or excreted from a human or animal body or any recovered fluid from a human or animal body, by any technique known to one skilled in the art such as extraction, sampling, draining, or washing. The steps for recovering and isolating or even encapsulating these different fluids from the human or animal body are achieved prior to the implementation.

Within the scope of the method according to the present invention, the plurality of targets cultured in a liquid medium in the culture chamber corresponds to a type of targets as described above (homogeneous population) or, alternatively, at least two different types of such targets (heterogenous population). The target Cᵢ, as for it, corresponding to any of the targets described above.

Within the scope of cultured in a liquid medium implies that the applied targets are found suspended in a suitable liquid medium, favorable to their survival and to the secretion of biomolecules. On this subject, the step for culturing in the method according to the present invention is understood under adapted or suitable conditions so that at least one compound is secreted by at least one target.

However it should be noted that some of these targets, when they are attached to a probe recognizing them and functionalizing one of the surfaces of the culture chamber, may in fact be found attached to the latter. Also, in the alternative method without functionalization of the 1^{st} surface by a probe, some of these targets may be deposited or settle on one of the surfaces of the culture chamber.

Further, the techniques and the conditions of cell culture notably in a liquid medium are well known to one skilled in the art, who will know how to define, for each type of target and notably each cell type, the nutritive, biological or synthetic, adequate medium, the optimum controlled cultivation temperature, for example 37° C., for many cells of mammals, as well as the atmosphere required for maintaining the viability of the cells, for example 5% CO₂. The culture period is also adaptable for each type of target and notably each type of cell, depending on its secretion rate. Generally, the culture period will be less than 24 h. Further, the culture may be stopped as soon as the sought information, i.e. the detection of a secreted compound and the determination of the secreting target will have been able to be obtained.

The probe able to bind specifically to the target Cᵢ is any molecule capable of forming with the target Cᵢ a binding pair, the probe and the target Cᵢ corresponding to both partners of this binding pair. The bonds applied in the probe-target Cᵢ binding are non-covalent bonds and with low energy such as hydrogen bonds or Van der Waals bonds, or bonds of higher energy of the covalent bond type.

The used probe is therefore dependent on the target Cᵢ to be detected. Depending on this target Cᵢ, one skilled in the art will know, without any inventive effort, how to select the most suitable probe. The probe may be any 1^{st} surface itself adapted to the attachment of targets such as a surface in at least one polymer adapted to the attachment of targets. Thus, the probe may be selected from the group formed by a peptide; an oligopeptide; a protein; a glycoprotein; an oligosaccharide; a polysaccharide; a carbohydrate; a lipoprotein; a lipid; a phospholipid; an agonist or antagonist of a membrane receptor; a polyclonal or monoclonal antibody; an antibody fragment such as a fragment Fab, F(ab′)₂, Fv, scFv, diabody or a hypervariable domain (or CDR for “Complementarity Determining Region”); a nucleotide molecule; a peptide nucleic acid; an aptamer such as a DNA aptamer or an RNA aptamer and a polymer adapted to the attachment of these targets such as a polymer of the poly(N-isopropylacrylamide) (pNIPAM) type, a polymer of the pNIPAM-co-acrylamidophenylboronate (pNIPAM-co-APBA) type, a polypyrrol, a polylysine, a polycyclic aromatic hydrocarbon (PAH), a polyetherimide (PEI) or a polyacrylic acid (PAA) . . . .

The expression “nucleotide molecule” used in the present invention is equivalent to the following terms and expressions: “nucleic acid”, “polynucleotide”, “nucleotide sequence”, “polynucleotide sequence”. By “nucleotide molecule” is meant within the scope of the present invention a regulating polynucleotide; an either single-strand or dual-strand, genomic, chloroplastic, plasmid, mitochondrial, recombinant or complementary DNA; a sequence acting as an aptamer; a portion or fragment thereof.

As an illustrative and non-limiting example, in the case when the target Cᵢ is a circulating cell, the probe may be selected from among the anti-CD (for “Clusters of Differentiation”) antibody.

The compound Cₒ secreted which one desires to track in real time the production thereof in the method according to the present invention may be any compound which may secrete one of the targets as previously defined under either natural conditions or not. In the latter case, the secretion of the compound Cₒ may be induced by particular conditions such as a stress or an infection.

Advantageously, the compound Cₒ is or comprises an element selected from the group consisting of a protein, a polypeptide, a peptide, a lipid, a glycoprotein, a glycolipid, a lipoprotein, ab inorganic ion, a small organic molecule comprising from 1 to 100 carbon atoms and a particulate or supramolecular compound, such as a vesicle, an exosome, a microbial organism such as for example a virus or a microbial particle such as for example a viral particle.

More particularly, the compound Cₒ is selected from the group consisting of a hormone, a pro-hormone, a neurotransmitter, a cytokine, a chemokine, a protein of the extra-cellular matrix, an immunoglobulin, a toxin, an infectious agent, such as a bacterium, a virus, or a protozoan parasite, or a constituent or production of an infectious agent, such as a viral protein or particle or a bacterial toxin.

The ligand used for functionalizing the 2^{nd} surface of the culture chamber implemented within the scope of the present invention is any molecule capable of forming with the compound Cₒ to be detected a binding pair, the compound Cₒ and the ligand corresponding to both partners of this binding pair. The bonds applied in the ligand-compound Cₒ binding are either non-covalent bonds with low energy such as hydrogen bonds or Van der Waals bonds, or strong energy bonds of the covalent bond type.

The ligand used therefore depends on the compound Cₒ to be detected. Depending on this compound, one skilled in the art will know, without any inventive effort, how to select the most suitable ligand. It may be selected from the group consisting of a peptide; an oligopeptide; a protein; a glycoprotein; an oligosaccharide; a polysaccharide; a carbohydrate; a lipoprotein; a lipid; a phospholipid; a polyclonal or monoclonal antibody; an antibody fragment such as a fragment Fab, F(ab′)₂, Fv, scFv, diabody or a hypervariable domain (or CDR for “Complementarity Determining Region”); a haptene; a nucleotide molecule as previously defined; a peptide nucleic acid; an aptamer such as a DNA aptamer or an RNA aptamer, a polymer adapted to the specific binding of inorganic ions, such as those described by Lange et a! (2008) [14] and a polymer adapted to the binding of these targets such as a polymer of the poly(N-isopropylacrylamide) (pNIPAM) type, a polymer of the pNIPAM-co-acrylamidophenylboronate (pNIPAM-co-APBA) type, a polypyrrol, a polylysine, a polycyclic aromatic hydrocarbon (PAH), a polyetherimide (PEI) or a polyacrylic acid (PAA).

As an illustrative and non-limiting example, a ligand which may be used within the scope of the method according to the present invention may be any antibody used in commercial ELISA kits.

The method according to the present invention wherein the 1^{st} surface is not functionalized by at least one probe recognizing at least one target Cᵢ is not only based on the use of a particular binding pair i.e. the ligand-compound Cₒ binding pair but also on a spatial arrangement of the targets and ligands, particularly in the culture chamber.

Alternatively, the method according to the present invention wherein the 1^{st} surface is functionalized by at least one probe recognizing at least one target Cᵢ is not only based on the use of two particular binding pairs i.e., on the one hand, the probe-target Cᵢ binding pair and, on the other hand, the ligand-compound Cₒ binding pair but also on a spatial arrangement of the ligands and probes, in particular in the culture chamber.

Indeed, in the methods according to the present invention, the 2^{nd} surface functionalized by ligands is different from the 1^{st} surface optionally functionalized with probes and non-coplanar with this 1^{st} surface. This characteristic explicitly excludes that the 1^{st} surface and the 2^{nd} surface are found together at a same surface of a non-physically structured solid support. In other words, the 1^{st} surface and the 2^{nd} surface do not correspond to distinct regions of the distributed spot type on one of the surfaces of a non-physically structured solid support. Thus, the 1^{st} and the 2^{nd} surfaces are physically distinct.

However, the 1^{st} surface and the 2^{nd} surface are found at a distance such that on the one hand, the signal emitted by the presence of the target Cᵢ on the 1^{st} surface or by the specific binding between the probe and the target Cᵢ (designated herein by 1^{st} signal) is distinguished from the signal emitted by the specific binding between said ligand and said compound Cₒ (designated herein by 2^{nd} signal) and that, on the other hand, it is the compound Cₒ secreted by a target Cᵢ present on the 1^{st} surface and optionally bound to a corresponding specific probe, which binds to a given ligand. In other words, the 1^{st} signal and the 2^{nd} signal are distinguished from one another, i.e. they do not interfere with each other and/or are not superposed on each other. Alternatively, at least one of the signals is independent thereby giving the possibility of differentiating the origin of the signals.

Further, the method and the device according to the present invention implement a specific spatial organization of the target(s) Cᵢ and of the specific ligand(s) of the compound Cₒ, in the case when the 1^{st} surface is non-functionalized. Alternatively, the method with functionalization of the 1^{st} surface and the device according to the present invention implement a specific spatial organization of the specific probe(s) of the target Cᵢ and of the specific ligand(s) of the compound Cₒ. There exists a proximity between these targets and these ligands or between these probes and these ligands so as to guarantee that the compound Cₒ secreted by a target Cᵢ optionally bound to a specific probe is attached to a given ligand. Thus, the 1^{st} surface and the 2^{nd} surface are spatially organized so as to be able to assign with certainty the cell origin of the type of detected secretion when the secreted molecules diffuse in the immediate environment of the targets. More particularly, the target and the ligand are distant from each other from 100 nm to 500 μm and advantageously by at least 300 nm. When the 1^{st} surface comprises several targets Cᵢ and when the 2^{nd} surface comprises several specific ligands of the compound Cₒ, each probe is distant from each target by at least 100 nm. Alternatively, the probe and the ligand are distant from each other from 100 nm to 500 μm and advantageously by at least 300 nm. When the 1^{st} surface comprises several specific probes of the target Cᵢ and when the 2^{nd} surface comprises several specific ligands of the compound Cₒ, each probe is distant from each ligand by at least 100 nm.

In a 1^{st} embodiment, the 1^{st} surface and the 2^{nd} surface belong to two different solid supports present in the culture chamber. Advantageously, both of these solid supports are placed facing each other and are separated by a space, typically filled with a culture medium as previously defined, from 10 to 500 μm. As examples, both of these solid supports may be two walls of the culture chamber, placed facing each other. The FIG. 3 hereafter relates to this 1^{st} embodiment.

In the latter, both of these solid supports may be of an identical or different nature. The nature of one of both solid supports is function of the detection technique used for detecting the 1^{st} signal, while that of the other solid support is function of the detection technique used for detecting the 2^{nd} signal.

In this configuration, several faces or solid supports are either capable of receiving a target, or functionalized by a specific ligand type of a secretion. The detection of the presence of the targets on certain faces or solid supports and of the ligand-compound interactions on other faces or solid supports may be accomplished either with the same technique, or with different techniques.

Alternatively, in this configuration, several faces or solid supports are functionalized either with a probe type specific of a target, or with a ligand type specific to a secretion. The detection of the interactions on these faces or solid supports may be accomplished either by the same technique, or by different techniques.

In this 1^{st} embodiment, it is also possible to functionalize the 2^{nd} surface, facing the 1^{st} surface functionalized with probes specific of a target Cᵢ, with several types of ligands specific of different compounds Cₒ. Each type of ligands is advantageously grouped in the form of a discrete region of affinity i.e. having affinity for a given compound Cₒ.

In a 2^{nd} embodiment, the 1^{st} surface and the 2^{nd} surface depend on a same solid support such as the bottom of the culture chamber. This 2^{nd} embodiment admits different alternatives.

In this 2^{nd} embodiment, the solid support is physically structured. By “physically structured”, is meant a support having a physical structuration involving embossed elements of the microstructure, pad, pillar, well type or of any other geometrical shape selected advantageously such as particles or beads the surface of which is able to receive targets or has an affinity for certain targets, and deposited on a surface having affinity for secretions.

FIGS. 1 and 2 hereafter relate to a 1^{st} alternative of this 2^{nd} embodiment, this alternative dealing with microstructures, plots, pillars and wells.

In this alternative, the physical structuration allows the generation of areas in depth (i.e. bottoms of wells or areas of the solid support surrounding the microstructures, plots or pillars) and of areas in height (i.e. upper edge of the wells or distal end of the microstructures, plots or pillars, the proximal end being in contact with the solid support). The surface of the areas in depth form one or several 2^{nd} surface(s) as previously defined and is therefore functionalized with ligands specifically binding to at least one compound Cₒ. Also, the areas in height correspond to one or several 1^{st} surface(s) as previously defined, these areas in height may optionally be functionalized with probes specific in binding to at least one target Cᵢ.

Advantageously, these embossed elements of the microstructure type have a height comprised between 100 nm and 10 μm and are advantageously spaced apart from each other by 1 nm to 10 μm, said spacing may be constant or variable.

In this 1^{st} alternative of the 2^{nd} embodiment, the solid support and the microstructure(s) may be of identical or different nature. The nature of the microstructure(s) depends on the detection technique used for detecting the 1^{st} signal, while that of the solid support depends on the detection technique used for detecting the 2^{nd} signal.

This alternative may give the possibility of producing a multiplexed method and device. Indeed, one or several microstructures close to each other may be functionalized with a probe specific of a target type and notably a cell type, thereby defining an area of affinity for such a target type or cell type and the solid support may have different microstructures defining different areas of affinity, each being specific to a given target or cell type. Also, the surface of the solid support may have different affinity regions for different compounds Cₒ.

In a 2^{nd} alternative of this 2^{nd} embodiment, one or several particle(s) are positioned on a solid support having at least one 2^{nd} surface as previously defined, all or part of the surface of at least one particle corresponding to a 1^{st} surface as previously defined.

By “particle”, is meant a solid particle, spherical or substantially spherical notably of the bead type, having a diameter comprised between 500 nm and 50 μm, said particle being optionally porous. When several particles are applied, it may be advantageous to use monodispersed particles or mixtures of different types of particles, each type of particles being monodispersed and this, in order to obtain a homogeneous deposit and thereby a homogeneous 1^{st} signal as previously defined.

In this alternative, the particles applied are not platonic solid with complete filling of the space between particles. Thus, the empty spaces between the particles generate pores which gives the possibility to the compounds secreted by the targets deposited or attached to the surface of such particles to travel towards the ligands functionalizing the surface of the underlying solid support. In other words, this alternative corresponds to an assembly of solid particles the organization of which generates pores. Thus, the assembled solid particles form a porous layer giving the possibility of separating or filtering the targets from the compounds to be detected.

Therefore, in this alternative, several solid particles are positioned on a solid support having at least one 2^{nd} surface as previously defined, all or part of the surface of at least one of these solid particles corresponding to a 1^{st} surface as previously defined and these solid particles forming a porous layer.

Advantageously, the size of the pores is less than ⅓ of the diameter of the particles. More particularly, the size of the pores is comprised between 100 and 500 nm whereby the pathway of the secreted compounds through the ligands functionalizing the surface of the underlying solid support is possible. The use of particles for which the organization generates pores may, inter alia, increase the sensitivity of the method and of the device by reducing the space occupied by the particles which may further be porous. Further, when the implemented particles are porous, the compounds may also travel via the open porosity of the latter.

Further, in this 2^{nd} alternative of the 2^{nd} embodiment, it is possible to implement particles having on one side functions allowing attachment to the surface of the underlying solid support and on the other side probes as previously defined. Such particles with two distinct faces are known as Janus particles.

In this alternative, it is possible to assemble particles with identical or different nature and having different functionalizations and to control their position on the surface of the underlying solid support thereby generating areas of affinity, the latter also having discrete regions functionalized by ligands specific of different secreted compounds Cₒ (affinity regions). Thus, it is possible, at the deposition, to obtain surfaces having sensitized patterns for detecting different substances and cells. The possibility of controlling the spatial localization of different functions on the surface at the deposition step is a clear advantage for marketing a device for multi-component detection. This advantage may considerably facilitate the biochemical functionalization required for the specificity of the device and this, already simply by reducing the number of preparation steps.

In such an assembly, the particles may appear as a monolayer or as a superposition of monolayers. Advantageously, the particles appear as a monolayer of the compact film type of particles.

One skilled in the art is aware of different techniques giving the possibility of producing deposits of a compact film of particles, the most known being:
the Langmuir-Blodgett method which implements a carrier liquid (for example water) in which is immersed beforehand in a vertical position the solid substrate, i.e. the “target” substrate on which has to be transferred the monolayer of particles. The particles are dispensed at the surface of the liquid on which they disperse. A mechanical barrier is then set into motion for gradually reducing the surface occupied by the particles in order to compress them. When the compact film is formed, the substrate is set into motion for depositing by capillarity the film at its surface. The barrier should accompany this drawing movement in order to preserve the compression of the particles [15],
by centrifugal coating, more known under the term of “spin coating” [16];
by the soaking-withdrawal technique, more known under the term of “dip-coating” a technique known to one skilled in the art; and
more particularly by an original method from the CEA [17] explained in the experimental part hereafter.

This 2^{nd} alternative of the 2^{nd} embodiment has many advantages, i.e. (i) a quasi-ideal geometry with a minimization of the contact between the solid support and the particles giving an optimal architecture so that the device notably using detection by surface plasmon resonance (SPR) does not lose any sensitivity; (ii) a perfect control of the assembling and of the size of the particles which gives the possibility of obtaining a reproducibility of the system and a fast, low cost deposition technology, efficient on voluminous objects of a complex geometry; and (iii) regular and known interstices, more comfortable than for a stochastic porosity (of the gel sol type).

Further, such a device is sufficiently robust so as to be handled in different solvents such as for example ethanol, acetone, toluene or water for its chemical functionalization, or under a hydrodynamic flow for rinsing and its use in microfluidics. Finally, it is biocompatible giving the possibility of carrying out biological studies in vitro.

Regardless of the considered embodiment or alternative, the implemented surfaces may have different areas or regions of affinity as previously defined. The number of probes or of different ligands immobilized on each of these areas or regions may for example range from 1 to 1,000 and notably from 1 to 500. The size of the areas or regions may be nanometric, micrometric, millimetric or centimetric.

Also, regardless of the considered embodiment or alternative, the implemented solid supports, the embossed elements, the particles, the 1^{st} surface and/or the 2^{nd} surface as previously defined may be solid supports, embossed elements, particles or surfaces of an organic material, of an inorganic material or of a mixture of at least one organic material and of at least one inorganic material.

As examples of a mixture, it is possible to contemplate the case when at least one surface selected from said 1^{st} surface and said 2^{nd} surface is in an organic material, the other surface being in an inorganic material.

Advantageously, the inorganic material is selected from the group consisting of glasses, quartzes, ceramics (for example, of the oxide type), metals (for example, platinum, aluminium, chromium, copper, zinc, silver, nickel, tin or gold), metalloids (for example, silicon or oxidized silicon), allotropic carbons (for example, glassy carbon, graphite, graphene, carbonaceous nanostructure of the nanoparticle or carbon nanotube type, or diamond) and mixtures thereof.

When the material of the solid supports, of the embossed elements, of the 1^{st} surface and/or of the 2^{nd} surface is organic, it advantageously corresponds to a polymeric material or an organic resin like for example agarose, a polyamide (of the nylon type), a polycarbonate, a polyethylene glycol, a fluoropolymer, an acrylate, a siloxane (polydimethylsiloxane; PDMS), a cyclic olefinic copolymer (COC), a polyether-ether-ketone (PEEK) or nitro-cellulose.

The functionalization by a probe and a ligand as previously defined of the 1^{st} surface and of the 2^{nd} surface respectively as previously defined may be carried out with any suitable technique allowing the attachment of a compound on a solid support. In particular, simple adsorption may be contemplated of ion bonds, hydrogen bonds, electrostatic interactions, hydrophobic interactions, Van der Waals bonds or covalent grafting.

In a particular embodiment of the present invention, the 1^{st} or the 2^{nd} surface has functional groups by means of which the probe(s) or the ligand(s) is(are) capable of being immobilized. Advantageously, these functional groups are selected from among carboxylic groups, radical entities, alcohol, amine, amide, epoxy or thiol functions. These groups may be intrinsic to the nature of the material of the 1^{st} or of the 2^{nd} surface. Alternatively, the latter may be obtained by cleaning said 1^{st} or 2^{nd} surface via at least one solvent, detergent, radiation or oxygen plasma or any other method allowing the formation of functional groups as previously defined.

In a first alternative of the present invention, the probe(s) may be immobilized directly at the 1^{st} surface either functionalized or not. A “coated” solid support with a protein is an example of direct immobilization.

In a second alternative of the present invention, the probe(s) may be immobilized indirectly at the 1^{st} surface either functionalized or not. This indirect immobilization involves a spacer arm (or junction agent) bound on the one hand to the 1^{st} surface and on the other hand to a probe. One skilled in the art is aware of different examples of such spacer arms. In a non-exhaustive way, mention may be made, as spacer arms which may be implemented within the scope of the present invention, 1,6 diaminohexane, 6-aminohexanoic acid, a silane, a succinimide group, an epoxide, a UDP-glucuronic acid, linear or branched alkyl chains from 1 to 20 carbon atoms, polyethylene glycol, glutaraldehyde, etc. . . . . All what has been described on the 1^{st} surface and the probes applies mutatis mutandis to the 2^{nd} surface and to the ligands.

This indirect immobilization applied to the device comprising solid particles positioned on a solid support implies that said solid support is chemically functionalized in order to allow the covalent or non-covalent assembly of said solid particles.

As illustrative examples, mention may more particularly be made of silane reagents applied for grafting of ligand(s) or probe(s) on glass, the complexing of thiol products on gold surfaces and the immobilization of ligand(s) or probe(s) in polymer matrices.

It should be noted that the preliminary immobilization on the support comprising the 1^{st} surface of a spacer arm may not only be implemented for allowing the functionalization by one or several ligand(s) but also for allowing the hooking-up of solid particles as previously defined on this support. A particular example of such a spacer arm is a self-assembled monolayer (SAM) of mercaptopropyltriethoxysilane or mercaptopropyltrimethoxysilane (MPTS) notably used on gold surfaces.

The bindings implemented during direct or indirect immobilization may be any bonds known to one skilled in the art and notably covalent bonds, ionic bonds, hydrogen bonds, electrostatic interactions, hydrophobic interactions, Van der Waals bonds, an adsorption, etc. . . .

Within the scope of the present invention, the 1^{st} (or the 2^{nd}) signal is produced during and/or from the deposit of the target Cᵢ on said 1^{st} surface or during and/or from the binding of the target Cᵢ to the probe (or of the compound Cₒ to the ligand) and may be measured by an element for measuring the signal. The detection or measurement of a signal refers to the determination of the presence or the absence of the 1^{st} (or of the 2^{nd}) signal, and/or its quantification. The expression in “real time” means that the 2^{nd} signal is produced and essentially measured at the moment when the binding of the compound Cₒ with the ligand takes place.

As already explained, the spatial organization applied between the 1^{st} surface and the 2^{nd} surface give the possibility of guaranteeing that, when a compound Cₒ is detected on the 2^{nd} surface, the secreting target is found at the 1^{st} surface and optionally bound to a probe. The measurement of the 1^{st} signal is therefore not mandatory but forms a control measure for purposes of verification.

The measurement of the 2^{nd} signal according to the invention is directly carried out. Thus, the signal does not require the mediation of molecules present in the culture chamber or added to the culture chamber, other than the compound Cₒ and the ligand, in order to be produced. As an example, the signal according to the invention does not stem from an oxidation-reduction probe, or from the additional binding of a marker specific to the compound Cₒ, such as a marked antibody, on a compound Cₒ already attached by the ligand. All which has been explained on the measurement of the 2^{nd} signal applies mutatis mutandis to the measurement of the 1^{st} signal.

Advantageously, any technique for detecting in real time and notably any technique for tracking the interaction reaction in real time known to one skilled in the art may be used for measuring the 2^{nd} signal. More particularly, this detection technique is selected from the group consisting of surface plasmon resonance in a single-point mode, the surface plasmon resonance in imaging, an optical technique in a near field and the measurement of impedance. Further information on the techniques implying a surface plasmon resonance may be obtained in the patent application US 2013/137085 [12].

As regards the detection of the 1^{st} signal, the latter advantageously involves an optical technique such as for example optical microscopy, optical microscopy in a guided mode or fluorescence microscopy. However, other modes of optical imaging may also be implemented for detecting the 1^{st} signal.

It should be noted that both the detection of the 1^{st} signal and that of the 2^{nd} signal requires at least one element for transducing this 1^{st} signal or this 2^{nd} signal able to transmit in real time the 1^{st} or the 2^{nd} signal produced by the presence of an object or by the binding of a type of object (target Cᵢ or compound Cₒ) on the specific affine area i.e. functionalized by a specific ligand or by a specific probe. Thus, an element for transducing the signal (measurement of the sole 2^{nd} signal) or of two elements for transducing the signal, either identical or different, are implemented within the scope of the present invention. An element for transducing the signal is any element allowing a real time detection by doing without the presence of mediators/developers whatever they are. It is advantageously selected from the group consisting of an impedance analyzer, a resonant beam and an optical readout system such as a surface plasmon resonance imager, a system for optical readout by optical microscopy or an optical readout system by fluorescent microscopy.

The present invention also relates to certain of the devices which may be implemented in a method as previously defined. These devices have been described previously and more particularly correspond,
on the one hand to the device comprising a culture chamber in which the 1^{st} surface and the 2^{nd} surface belong to two different solid supports and,
on the other hand, to the device as previously defined, comprising a culture chamber in which one or several particle(s) are positioned on a solid support having at least one 2^{nd} surface as previously defined, all or part of the surface of at least one particle corresponding to a 1^{st} surface as previously defined.

The method and the devices according to the present invention have many applications in which it is necessary to measure the secretion or the release of one or several compounds by one or several given secreting cell targets.

One of the general fields of application of the method and of the devices according to the invention deals with technologies for healthcare, and more particularly for applications turned towards the analysis of biological fluids as previously defined, and also for diagnostic applications. As illustrative and non-limiting examples of such applications, mention may be made of:
the evaluation of vaccinal coverage (HBV test, influenza test and Tuberculosis test);
the characterization of immuno-depressed conditions (congenital or acquired like AIDS or resulting from treatments like in organ grafts);
the characterization of auto-immune diseases (diabetes, lupus, multiple sclerosis etc.) and
preliminary tests of graft compatibility.

Another field of application of the method and of the devices according to the present invention consists in their use in the pharmaceutical industry, notably in screening projects of molecules with an immuno-modulator potential, as a stimulant like in the scope of vaccines, or depressing like in the scope of anti-inflammatories. As the question is in these cases of identifying molecules which may trigger a controlled immune reaction, the present invention seems particularly adapted for rapidly detecting and accurately detecting (kinetic response of individual cells) and in a rapid way any molecule regulating the immune system. For this purpose, the molecule to be tested is present in the culture medium and the measurement of the secretion of a compound Cₒ by a secreting target C obtained in the presence of said molecule to be tested is compared with the secretion of the same compound Cₒ by a same secreting target Cᵢ obtained in the absence of this molecule.

Another more academic application of the method and of the devices according to the invention may consist in studying the influence of a given condition on a target Cᵢ and notably on the secretion of the compound Cₒ secreted by the latter.

Advantageously, the condition which one seeks to determine the influence on the target Cᵢ and on its secretion is a physical, chemical or biological condition.

By “physical condition”, is meant a physical condition which modifies the environment in which is found the target Cᵢ such as a thermal condition (modification of the temperature of the culture medium), an electric or electromagnetic condition (environment and therefore target Cᵢ subjected to electric stimulation or to an electromagnetic wave), a mechanical condition or a radioactive condition.

By “chemical condition” is meant a chemical condition which modifies the environment in which is found the target Cᵢ such as the addition of a compound to be tested in the culture medium and/or the modification of its concentration, the modification of the nature and/or of the concentration of the ions contained in said culture medium.

By “biological condition”, is meant a condition of a biological nature which modifies the environment in which is found the target Cᵢ such as the presence of cells belonging to another cell type, bacteria, archaeae, parasites, fungi, yeasts or viruses, or the presence of biomolecules (chemokines, antigen fragments, change of culture medium . . . ).

Also in this application, the measurement of the secretion of a compound Cₒ by a secreting target Cᵢ is compared in the presence or in the absence of the condition to be tested.

The invention will be better understood upon reading the figures and examples which follow. The latter do not have the purpose of limiting the invention in these applications, the only purpose is to illustrate here the possibilities provided by the method and the devices of the invention.

### SHORT DESCRIPTION OF THE DRAWINGS

FIG. 1 shows an exemplary application with probes and ligands deposited on the same solid support. The solid support is structured (FIG. 1A) so as to show highly functionalized regions with specific probes of cell types (regions in contact with the cells) (FIG. 1B) as well as low regions (here at the bottom of the wells) for capturing by means of specific probes the secreted molecules and thereby detect them with an evanescent wave (FIG. 1C) during activation of the cell secretion phenomenon (FIG. 1D).

FIG. 2 shows two embodiments of structured domains at the surface of a same solid support, either by pads which correspond to a configuration identical with that of FIG. 1 (FIG. 2A), or by wells (FIG. 2B).

FIG. 3 shows an example of the mounting of a device provided with two functionalized faces, one (lower material) dedicated to the capture of cells on specific affinity regions and the other (material above) favorable to the detection of secreted molecules (and diffusing from the cells immobilized in proximity) by ligands for which the binding to their target is tracked by surface plasmon resonance.

FIG. 4 shows the schematized deposit of differently pre-functionalized beads for immobilization of differentiated cells.

### DETAILED DISCUSSION OF PARTICULAR EMBODIMENTS

1. Devices Implemented in the Method According to the Invention

These devices have a structured face. Indeed, in order to distinguish the signal due to the targets and that due to the secretions, certain studies at the basis of the present invention aimed at structuring the analysis surface in order to move away the cell targets. Let us note that the dimension of the cells (1 to 10 μm) is generally greater by several orders of magnitude to the secretions (1 to 100 nm).

More particularly, the readout of the present biosensor is accomplished by means of surface plasmon resonance imaging (SPR) for secretions and by another mode like optical microscopy for the cells, the device 1 contains a surface structuration as follows:
regions “in depth” 2 of the device making up one or several 2^{nd} surface(s) as previously defined are functionalized with ligands 3 specific of the secretions. This may for example be anti-cytokine antibodies deposited on a gold surface, for which the complexing with the antigen i.e. the cytokine produces an imaging signal SPR; and
regions “in height” 4, i.e. at an altitude above 100 nm in order to be located out of the field of the propagation of the evanescent wave generated by the surface plasmons. These domains correspond to one or several 1^{st} surface(s) as previously defined are functionalized by probes 5 specific of different cell types. The size of these domains is greater than the size of an individual cell (i.e. about 10 microns in diameter), and may attain several square millimeters, or even one square centimeter.

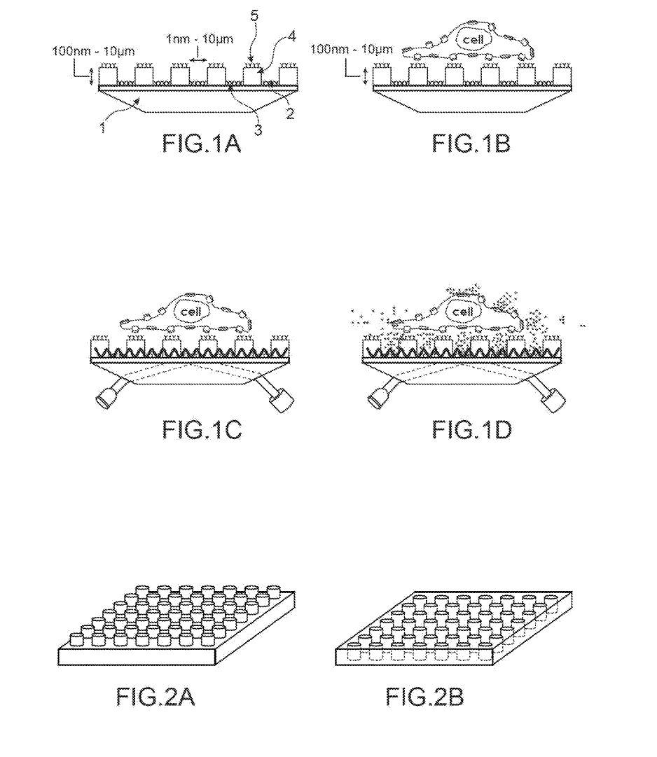

In order to dissociate the signals detected by SPR due either to the capture of the cells, or to that of the secreted biomolecules, the application of a device for which a sensitive face of the biosensor is structured may be contemplated. The propagation of the evanescent wave produced by the plasmon resonance occurs on about 100 nm above the metal film. Several functionalized regions, at different “altitudes” above the sensitive film of the biosensor may be made for example, by specific capture of cells in height (i.e. out of the field of SPR detection), and the capture of the secretions at low altitude (in the field of the SPR detection) according to FIG. 1.

The supports containing the binding partners of the cells and of the secreted compounds may either be pillars for which the apices are functionalized with probes specifically binding to one or several cell types and the spaces between the pillars by ligands specifically binding to one or several types of compound(s) (FIG. 2A), or a planar surface functionalized by probes specifically binding to one or several cell types, and perforated right through for allowing functionalization of the wells thereby formed by ligands specifically binding to one or several types of compounds (FIG. 2B).

However, the techniques applied for this structuration and the devices finally obtained may, in certain cases, have one or several drawbacks selected from among a very slow making and therefore impossible to industrialize, a loss of sensitivity, a fragility of the device notably in biological media and in materials which are difficult to characterize.

2. Devices According to the Present Invention

### 2.1. Device Containing Two Functionalized Faces.

This circuit contains two faces 6 and 7, functionalized and placed facing each other, separated by a spacer from 10 to 500 μm. This spacer filled with culture medium allows the diffusion of the secretions from the cell targets captured on one face towards the ligands immobilized on the other one.

One of the faces 6 which may for example be a glass slide, a fine gold film or a polymeric material is functionalized with probes 8 specific of targets and the other face 7 is functionalized by means of ligands 9 specific of cell secretions 10.

This latter face is compatible with SPR imaging in order to track the capture of the molecules secreted by the captured cells on the opposite face and may be a glass support covered with a gold film for example.

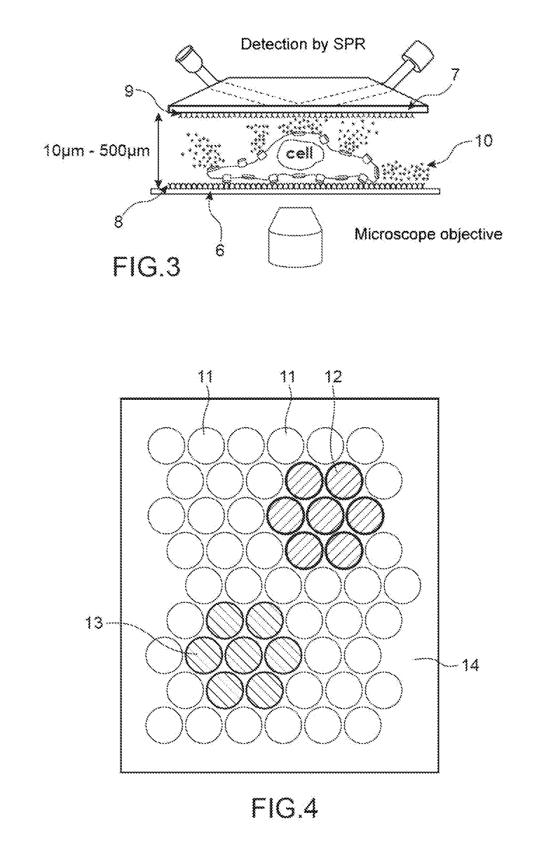

In the circuit described in FIG. 3, the face dedicated to the cell capture is coupled with an optical detection by conventional microscopy on the lower face while the secreted biomolecules diffusing in the medium are captured on the functionalized upper face by specific ligands and detected by SPR. In this configuration, each face of observation simultaneously produces different pieces of information, either dealing with the specific capture of the cells or of the specific capture of the secretions.

2.2. Device with a Carpet of Nano-Microspheres.

In this device, the solid support for which the surface is functionalized with ligands specifically binding to secreted molecules is a glass prism Horiba—SF11 (n=1.71, high optical quality), for which the planar surface was coated with 47 nm of gold on 3 nm of titanium for adhesion on glass [18, 19].

Further, in order to allow adhesion of the silicon beads on gold, a self-assembled monolayer of mercaptopropyltriethoxysilane or mercaptopropyltrimethoxysilane (MPTS) is formed on the golden surface. This treatment was achieved by using a bath with 10 mM of MPTS in toluene for 12 h, followed by abundant rinsings with toluene, ethanol and water and then by drying at room temperature under a flow of inert gas.

The spherical silica particles may consist of a mixture consisting of 25% in number of particles for which the average diameter is 500 nm and 75% in number of particles for which the average diameter is 1 μm. Indeed, a monolayer having such a proportion of particles of 500 nm and of 1 μm is more homogeneous in SPR imaging than a monolayer only composed of beads with a diameter of 1 μm.

Certain of the beads are functionalized with a type of antibody according to protocols well known to one skilled in the art such as for example the one described by Moon et al, 1996 [20]. It is also possible to assemble beads having different functionalizations and/or nature and of controlling their position on the surface. Thus, it is possible, at the deposition to obtain surfaces having sensitized patterns for detecting different substances and cells (as schematized in FIG. 4).

In FIG. 4, the beads 11 are not functionalized and the beads 12 and 13 are functionalized so as to specifically recognize lymphocytes B (anti-CD19 antibodies, CD19 protein, or otherwise called eB4, being a protein expressed at the cell membrane and considered as a marker of lymphocytes B) and lymphocytes T (anti-CD90 antibodies, CD90 protein, or further called Thy being a protein expressed at the cell membrane and considered as a marker of lymphocytes T) respectively. It should be noted that this specific detection brought by the functionalization of the beads 12 and 13 is to be cross-referenced with the specific recognition of substances by the functionalization of the gold of the support 14.

For this purpose, a compact film of beads is made on the gold surface of the solid support functionalized beforehand with a self-assembled monolayer (SAM) of mercaptopropyltriethoxysilane or mercaptopropyltrimethoxysilane (MPTS) and this, by using the method described in the international application WO 2014/037559 [17]. In this method, 3 parts are distinguished:

(i) a system allowing dispensing of the particles in solution. In order for the method to operate, the particles should imperatively float at the surface of a carrier liquid such as water;

(ii) a liquid conveyor for transporting and laying out the particles in order to form a compact film. This liquid conveyor flows on a tilted plane and then in a horizontal area called a transfer area. The binding between the carrier liquid and the substrate is ensured by a capillary bridge;

(iii) the solid support as prepared beforehand and functionalized by a self-assembled monolayer (SAM) of mercaptopropyltriethoxysilane or mercaptopropyltrimethoxysilane (MPTS) as previously described, on which the compact film has to be transferred and set into motion by a conveyor.

The method therefore consists of dispensing the particles at the surface of the carrier liquid. The carrier liquid carries the particles as far as the transfer area. The particles accumulate in the transfer area and then move up the tilted plane. The particles present on the tilted plane then exert pressure which contributes to ordering the particles present in the transfer area.

### REFERENCES

[1] International application WO 98/23960 in the name of Isis Innovation Ltd published on Jun. 4, 1998.
[2] Czerkinski et al, 1983, “A solid-phase enzyme-linked immunospot (ELISPOT) assay for enumeration of specific antibody-secreting cells”, J. Immunol. Methods, Vol. 65, pages 109-121.
[3] Prussin & Metcalfe, 1995, “Detection of intracytoplasmic cytokine using flow cytometry and directly conjugated anti-cytokine antibodies”, J. Immunol. Methods, Vol. 188, pages 117-128.
[4] Turcanu & Williams, 2001, “Cell identification and isolation on the basis of cytokine secretion: A novel tool for investigating immune responses”, Nat. Med., Vol. 7, pages 373-376.
[5] Manz et al, 1995, “Analysis and sorting of live cells according to secreted molecules, relocated to a cell-surface affinity matrix”, Proc. Natl. Acad. Sci. USA, Vol. 92, pages 1921-1925.
[6] Westerink & Ewing, 2008, “The PC12 cell as a Model for Neurosecretion”, Acta Physiol., Vol. 192, pages 273-285.
[7] Huang et al, 2011, “Micro- and Nanotechnologies for Study of Cell Secretion”, Anal. Chem., Vol. 83, pages 4393-4406.
[8] International application WO 2011/056643 in the name of The University of Michigan published on May 12, 2011.
[9] Wu et al, 2013, “Optofluidic Platform for Real-Time Monitoring of Live Cell Secretory Activities Using Fano Resonance in Gold Nanoslits”, Small, Vol. 9, pages 3532-3540.
[10] International application WO 2006/060646 in the name of The University of Texas System published on Jun. 8, 2006.
[11] Guedon et al, 2000, “Characterization and Optimization of a Real-Time, Parallel, Label-Free, Polypyrrole-Based DNA Sensor by Surface Plasmon Resonance Imaging”, Anal. Chem., Vol. 72, pages 6003-6009.
[12] Patent application US 2013/137085 in the name of the CEA published on May 30, 2013.
[13] Zhu et al, 2008, “A microdevice for multiplexed detection of T-cell-secreted cytokines”, Lab Chip, Vol. 8, pages 2197-2205.
[14] Lange et al, 2008, “Conducting polymers in chemical sensors and arrays”, Anal. Chim. Acta, Vol. 614, pages 1-26.
[15] Bardosova et al, 2010, “The Langmuir-Blodgett Approach to Making Colloidal Photonic Crystals from Silica Spheres”, Adv. Mater., Vol. 22, pages 3104-3124.
[16] Bauert et al, 2005, “Self-Assembling of Particle Monolayers by Spin-Coating”, European Cells and Materials, Vol. 10, suppl. 5, page BS2.
[17] International application WO 2014/037559 in the name of the CEA published on Mar. 13, 2014.
[18] Grosjean et al, 2005, “A polypyrrole protein microarray for antibody-antigen interaction studies using a label-free detection process”, Anal Biochem, Vol. 347, pages 193-200.
[19] Suraniti et al, 2007, “Real-time detection of lymphocytes binding on an antibody chip using SPR imaging”, Lab Chip, Vol. 7, pages 1206-1208.
[20] Moon et al, 1996, “Formation of uniform aminosilane thin layers: an imine formation to measure relative surface density of the amine group”, Langmuir, Vol. 12, pages 4621-4624.

1-20. (canceled)

21. A method for measuring in real time the secretion of a compound by a target, the method comprising:
culturing in a liquid medium, in a culture chamber, a plurality of targets among which is found at least one target, the culture chamber including:
i) at least one 1^{st} surface on which the target is present, presence of the target on the 1^{st} surface generating a 1^{st} signal, and
ii) at least one 2^{nd} surface, different from the 1^{st} surface and non-coplanar with the 1^{st} surface, functionalized with at least one ligand specifically binding to the compound secreted by the target, the specific binding between the ligand and the compound generating a 2^{nd} signal distinct from the 1^{st} signal; and
real time detecting the 2^{nd} signal and optionally detecting the 1^{st} signal.

22. The method according to claim 21, wherein the target and the ligand are distant from one another by 100 nm to 500 μm.

23. The method according to claim 21, wherein the target is a secreting element comprising one or plural identical or different cells.

24. The method according to claim 21, wherein the target is (i) an individual cell, (ii) a set of identical cells or a group of encapsulated identical cells, (iii) a set of cells of at least two different types such as a tissue or a tissue fragment, or (iv) a set of cells of at least two different encapsulated types.

25. The method according to claim 21, wherein the compound is an element selected from the group consisting of: a protein, a polypeptide, a peptide, a lipid, a glycoprotein, a glycolipid, a lipoprotein, an inorganic ion, a small organic molecule comprising from 1 to 100 carbon atoms and a particulate or supramolecular compound, a vesicle, an exosome, a microbial organism, a virus, a microbial particle, or a viral particle.

26. The method according to claim 21, wherein the ligand is selected from the group consisting of: a peptide; an oligopeptide; a protein; a glycoprotein; an oligosaccharide; a polysaccharide; a carbohydrate; a lipoprotein; a lipid; a phospholipid; a polyclonal or monoclonal antibody; an antibody fragment or a fragment Fab, F(ab′)₂, Fv, scFv, diabody or a hypervariable domain, or CDR for a Complementarity Determining Region; a haptene; a nucleotide molecule as previously defined; a peptide nucleic acid; an aptamer such as a DNA aptamer or an RNA aptamer, a polymer adapted to the specific attachment of inorganic ions and a polymer adapted to the attachment of these targets such as a polymer of the poly(N-isopropylaciylamide) (pNIPAM) type, a polymer of the pNIPAM-co-acrylamidophenylboronate (pNIPAM-co-APBA) type, a polypyrrol, a polylysine, a polycyclic aromatic hydrocarbon (PAH), a polyetherimide (PEI) or a polyacrylic acid (PAA).

27. The method according to claim 21, wherein the 1^{st} and the 2^{nd} surfaces belong to two different solid supports present in the culture chamber.

28. The method according to claim 21, wherein the 1^{st} and the 2^{nd} surfaces depend on a same solid support having a physical structuration involving embossed elements.

29. The method according to claim 21, wherein plural solid particles are positioned on a solid support having at least one 2^{nd} surface, all or part of the surface of at least one of the solid particles corresponding to a 1^{st} surface and the solid particles forming a porous layer.

30. The method according to claim 21, wherein the solid supports, the embossed elements, the particles, the 1^{st} surface and/or the 2^{nd} surface are solid supports, embossed elements, particles and/or surfaces in an inorganic material selected from the group consisting of: glasses, quartzes, ceramics, metals, metalloids, allotropic carbons, and mixtures thereof.

31. The method according to claim 21, wherein the solid supports, the embossed elements, the particles, the 1^{st} surface and/or the 2^{nd} surface are solid supports, embossed elements, particles and/or surfaces in an organic material, selected from the group consisting of: agarose, polyamide (nylon type), polycarbonate, polyethylene glycol, fluoropolymer, acrylate, a siloxane (polydimethylsiloxane; PDMS), a cyclic olefin copolymer (COC), a polyether-ether-ketone (PEEK) and nitro-cellulose.

32. The method according to claim 21, wherein at least one surface selected from between the 1^{st} surface and the 2^{nd} surface is in an organic material, selected from the group consisting of: agarose, a polyamide (nylon type), a polycarbonate, a polyethylene glycol, a fluoropolymer, an acrylate, a siloxane (polydimethylsiloxane; PDMS), a cyclic olefin copolymer (COC), a polyether-ether-ketone (PEEK) and nitro-cellulose, the other surface being in an inorganic material selected from the group consisting of: glasses, quartzes, ceramics, metals, metalloids, allotropic carbons and mixtures thereof.

33. The method according to claim 21, wherein a technique used for detecting the 2^{nd} signal is selected from the group consisting of: surface plasmon resonance in a single point mode, surface plasmon resonance in imaging, an optical technique in a near field and measurement of impedance.

34. The method according to claim 21, wherein a technique used for detecting the 1^{st} signal is selected from the group consisting of: an optical technique, optical microscopy, optical microscopy in a guided mode, or fluorescence microscopy.

35. A device which may be implemented in a method as defined in claim 21, the device comprising a culture chamber wherein
a 1^{st} surface optionally functionalized with at least one probe specifically binding to a target, and
a 2^{nd} surface, different from the 1^{st} surface and non-coplanar with the 1^{st} surface, functionalized by at least one ligand specifically binding to a compound secreted by the target optionally bound to the probe,
belong to two different solid supports.

36. A method for measuring in real time the secretion of a compound by a target, the method comprising:
culturing in a liquid medium in a culture chamber a plurality of targets among which is found at least one target, the culture chamber including:
i) at least one 1^{st} surface functionalized with at least one probe specifically binding to the target, the specific binding between the probe and the target generating a 1^{st} signal, and
ii) at least one 2^{nd} surface, different from the 1^{st} surface and non-coplanar with the 1^{st} surface, functionalized by at least one ligand specifically binding to the compound secreted by the target bound to the probe, specific binding between the ligand and the compound generating a 2^{nd} signal distinct from the 1^{st} signal; and
real time detecting the 2^{nd} signal and optionally detecting the 1^{st} signal.

37. The method according to claim 36, wherein the probe is selected from the group consisting of: a peptide; an oligopeptide; a protein; a glycoprotein; an oligosaccharide; a polysaccharide; a carbohydrate; a lipoprotein; a lipid; a phospholipid; an agonist or antagonist of a membrane receptor; a polyclonal or monoclonal antibody; an antibody fragment such as a fragment Fab, F(ab′)₂, Fv, scFv, diabody or a hypervariable domain or CDR for Complementarity Determining Region; a nucleotide molecule; a peptide nucleic acid; an aptamer such as a DNA aptamer or an RNA aptamer and a polymer adapted to the attachment of these targets such as a polymer of the poly(N-isopropylacrylamide) (pNIPAM) type, a polymer of the pNIPAM-co-acrylamidophenylboronate (pNIPAM-co-APBA) type, a polypyrrol, a polylysine, a polycyclic aromatic hydrocarbon (PAH), a polyetherimide (PEI) or a polyacrylic acid (PAA).

38. A device which may be implemented in a method as defined in claim 21, the device comprising a culture chamber wherein plural solid particles are positioned on a solid support and form a porous layer,
all or part of the surface of at least one of the solid particles corresponding to a 1^{st} surface optionally functionalized with at least one probe specifically binding to a target and
the solid support having at least one 2^{nd} surface, different from the 1^{st} surface and non-coplanar with the 1^{st} surface, functionalized by at least one ligand specifically binding to a compound secreted by the target optionally bound to the probe.

39. The device according to claim 38, wherein the solid support is chemically functionalized for allowing covalent or non-covalent assembling of the solid particles.

40. The device according to claim 38, wherein a spacer arm of the self-assembled monolayer type (SAM) of mercaptopropyltriethoxysilane or mercaptopropyltrimethoxysilane (MPTS) was immobilized beforehand on the solid support for giving the possibility of hooking-up the solid particles.
